(19) United States
(12) Patent Application Publication
(10) Pub. No.: US 2017/0079599 A1
(43) Pub. Date: Mar. 23, 2017

(54) MOISTURE FEELING EVALUATION DEVICE, MOISTURE FEELING EVALUATION METHOD, AND MOISTURE FEELING EVALUATION PROGRAM

(71) Applicant: FUJIFILM Corporation, Tokyo (JP)

(72) Inventors: Naoko YOSHIDA, Ashigara-kami-gun (JP); Ikuko OHGARU, Ashigara-kami-gun (JP); Eriko IKEDA, Ashigara-kami-gun (JP)

(73) Assignee: FUJIFILM Corporation, Tokyo (JP)

(21) Appl. No.: 15/365,193

(22) Filed: Nov. 30, 2016

Related U.S. Application Data
(63) Continuation of application No. PCT/JP2015/062116, filed on Apr. 21, 2015.

(30) Foreign Application Priority Data
Jul. 18, 2014 (JP) 2014-147400

(51) Int. Cl. A61B 5/00; G06T 7/90; A61B 5/103

(52) U.S. Cl. CPC A61B 5/7278; A61B 5/443; A61B 5/0059; A61B 5/103; G06T 7/90; G06T 2207/10024; G06T 2207/30201

(57) ABSTRACT

In a moisture feeling evaluation device according to the present invention, an image input unit 1 receives an input of a captured image obtained by imaging a face F of a subject for which making up is performed, a brightness-color index calculation unit 10 calculates all of the amount of generated gloss, the amount of stain portions, and the amount of color irregularity portions as brightness-color indexes based on the captured image input to the image input unit 1, a shape index calculation unit 11 calculates all of the amount of wrinkle portions and the amount of pore portions as shape indexes based on the captured image input to the image input unit 1, and a moisture feeling evaluation unit 5 evaluates a feeling of visible moisture of the face F of the subject for which making up is performed based on the brightness-color indexes and the shape indexes.

### CROSS-REFERENCE TO RELATED APPLICATIONS

This application is a Continuation of PCT International Application No. PCT/JP2015/062116 filed on Apr. 21, 2015, which claims priority under 35 U.S.C. §119(a) to Japanese Patent Application No. 2014-147400 filed on Jul. 18, 2014. The above application is hereby expressly incorporated by reference, in its entirety, into the present application.

### BACKGROUND OF THE INVENTION

1. Field of the Invention

The present invention relates to a moisture feeling evaluation device, a moisture feeling evaluation method, and a moisture feeling evaluation program, and particularly to a moisture feeling evaluation device, a moisture feeling evaluation method, and a computer-readable recording medium having a moisture feeling evaluation program that evaluate a feeling of moisture based on a captured image obtained by imaging the face of a subject.

2. Description of the Related Art

In recent years, in the cosmetics field, various methods of evaluating a feeling of moisture of the skin have been proposed. In general, skin having a feeling of moisture refers to fresh and youthful skin that looks like it contains moisture. However, when the amount of moisture or the like of the skin is simply measured to evaluate the feeling of moisture, it is not possible to obtain a high correlation with a visual evaluation in which an observer actually views the skin for evaluation. Accordingly, it is desirable to provide an evaluation method having a high correlation with visual evaluation.

As such a moisture feeling evaluation method, for example, as disclosed in JP2003-024282A, a skin state evaluation method of evaluating a feeling of moisture of the skin by associating a mean fractional coefficient and/or a fluctuation coefficient of the mean fractional coefficient on a surface of the skin of a person and a visual index indicating the state of the skin has been proposed. In this way, it is possible to evaluate the feeling of moisture with high accuracy by associating a physical characteristic of the skin of a person with a visual index for evaluation.

Further, in recent years, a demand for evaluating a feeling of moisture even with respect to skin on which making up has been applied (made-up skin) with high accuracy has increased. JP2003-024282A discloses a technique for evaluating a feeling of moisture of skin after being made up.

### SUMMARY OF THE INVENTION

However, in the evaluation method disclosed in JP2003-024282A, the feeling of moisture is evaluated based on partial physical characteristics of made-up skin, and thus, while it is possible to obtain a high correlation with respect to visual evaluation when partially observing the skin, it is not possible to obtain a high correlation with respect to visual evaluation for evaluating the feeling of moisture (feeling of visible moisture) when generally viewing the made-up skin. In reality, when evaluating the feeling of moisture, the feeling of visible moisture is evaluated by generally viewing the made-up skin. Thus, an evaluation result which is greatly different from a sensation of the feeling of moisture when actually viewing the made-up skin is obtained. In particular, in the case of the face, the state of the made-up skin complicatedly changes according to a position, and thus, it is difficult to obtain an evaluation result having a high correlation with the sensation of the feeling of moisture when actually viewing the made-up skin from evaluation of only a part of the made-up skin or from only one type of physical characteristic of the made-up skin.

In order to solve these problems, an object of the invention is to provide a moisture feeling evaluation device, a moisture feeling evaluation method, and a moisture feeling evaluation program capable of evaluating a feeling of visible moisture of the face of a subject for which making up is performed with high accuracy.

According to an aspect of the invention, there is provided a moisture feeling evaluation device comprising: an image input unit that receives an input of a captured image obtained by imaging a face of a subject for which making up is performed; a brightness-color index calculation unit that calculates a brightness-color index of the face of the subject for which making up is performed based on brightness values and color component values of the captured image input to the image input unit; a shape index calculation unit that detects uneven portions of the face of the subject for which making up is performed based on the brightness values and the color component values of the captured image input to the image input unit and calculates a shape index based on the amount of uneven portions; and a moisture feeling evaluation unit that evaluates a feeling of visible moisture of the face of the subject for which making up is performed based on the brightness-color index and the shape index, in which the brightness-color index calculation unit includes a gloss calculation unit that calculates the amount of generated gloss due to making up by setting a first evaluation region in a cheek portion of the face of the subject and setting a reference region in a portion surrounding the first evaluation region and calculating at least one of a difference between brightness representative values and a difference between saturation representative values of the color components in the first evaluation region and the reference region, a stain calculation unit that calculates the amount of stain portions by detecting the stain portions based on the brightness values or the color component values, and a color irregularity calculation unit that calculates the amount of color irregularity portions by detecting the color irregularity portions based on the brightness values or the color component values, and calculates all of the amount of the generated gloss, the amount of stain portions, and the amount of color irregularity portions as the brightness-color indexes, and the shape index calculation unit includes a wrinkle calculation unit that calculates the amount of wrinkle portions by detecting the wrinkle portions based on the brightness values, a pore calculation unit that calculates the amount of pore portions by detecting the pore portions based on the brightness values or the color component values, and calculates all of the amount of wrinkle portions and the amount of pore portions as the shape indexes.

Here, it is preferable that the gloss calculation unit calculates a difference between saturation average values or a difference between saturation minimum values in the first evaluation region and the reference region as the difference between the saturation representative values by calculating a saturation average value or a saturation minimum value with respect to each of the first evaluation region and the reference region.

Further, it is preferable that the gloss calculation unit calculates a difference between brightness average values or a difference between brightness maximum values in the first evaluation region and the reference region as the difference between the brightness representative values by calculating a brightness average value or a brightness maximum value with respect to each of the first evaluation region and the reference region.

Further, it is preferable that the stain calculation unit sets a second evaluation region in the face of the subject, detects the stain portions where the brightness value or the color component value locally changes, having a predetermined size, in the second evaluation region, and calculates a total area of the stain portions, a proportion of the area of the stain portions in the second evaluation region, shades of the stain portions in the second evaluation region, or the number of stain portions, as the amount of stain portions, and the color irregularity calculation unit sets a third evaluation region in the face of the subject, detects the color irregularity portions where the brightness value or the color component value is locally changed, having a size larger than that of each stain portion, from the third evaluation region, and calculates a total area of the color irregularity portions, an area ratio of the color irregularity portions with respect to the third evaluation region, shades of the color irregularity portions in the third evaluation region, and the number of color irregularity portions, as the amount of color irregularity portions.

Further, it is preferable that the wrinkle calculation unit sets a fourth evaluation region that extends from the nostrils to the corners of the mouth in the face of the subject, detects the wrinkle portions where the brightness value decreases in the fourth evaluation region, and calculates a total area of the wrinkle portions, an area ratio of the wrinkle portions with respect to the fourth evaluation region, shades of the wrinkle portions, or the lengths of the wrinkle portions, as the amount of wrinkle portions, and the pore calculation unit sets a fifth evaluation region in the face of the subject, detects the pore portions where the brightness value or the color component value is locally changed, having a size smaller than that of each stain portion, from the fifth evaluation region, and calculates a total area of the pore portions, an area ratio of the pore portions with respect to the fifth evaluation region, shades of the pore portions, and the number of pore portions in the fifth evaluation region, as the amount of pore portions.

Further, it is preferable that the moisture feeling evaluation unit calculates in advance a reference value of the feeling of visible moisture with respect to a linear sum of the brightness-color index and the shape index by visually evaluating faces of a plurality of subjects for which making up is performed, and evaluates the feeling of visible moisture based on the reference value.

Further, it is preferable that the moisture feeling evaluation device further includes a database that stores the reference value with respect to the linear sum of the brightness-color index and the shape index, and the moisture feeling evaluation unit calculates an evaluation value of the feeling of visible moisture with reference to the database based on the brightness-color index value calculated in the brightness-color index calculation unit and the shape index value calculated in the shape index calculation unit.

According to another aspect of the invention, there is provided a moisture feeling evaluation method comprising: receiving an input of a captured image obtained by imaging a face of a subject for which making up is performed; setting a first evaluation region in a cheek portion of the face of the subject, setting a reference region in a portion surrounding the first evaluation region, calculating a brightness representative value and a saturation representative value in each of the first evaluation region and the reference region based on brightness values of the captured image and saturation values of color components thereof, and calculating the amount of generated gloss due to making up by calculating at least one of a difference between the brightness representative values and a difference between the saturation representative values in the first evaluation region and the reference region; calculating the amount of stain portions by detecting the stain portions based on the brightness values or the color component values of the captured image; calculating the amount of color irregularity portions by detecting the color irregularity portions based on the brightness values or the color component values of the captured image; calculating the amount of wrinkle portions by detecting the wrinkle portions based on the brightness values of the captured image; calculating the amount of pore portions by detecting the pore portions based on the brightness values or the color component values of the captured image; and evaluating a feeling of visible moisture of the face of the subject for which making up is performed, using all of the amount of the generated gloss, the amount of stain portions, and the amount of color irregularity portions as the brightness-color indexes and using all of the amount of wrinkle portions and the amount of pore portions as the shape indexes, based on the brightness-color indexes and the shape indexes.

According to still another aspect of the invention, there is provided a non-transitory computer-readable recording medium that records a moisture feeling evaluation program that causes a computer to execute: a step of receiving an input of a captured image obtained by imaging a face of a subject for which making up is performed; a step of setting a first evaluation region in a cheek portion of the face of the subject, setting a reference region in a portion surrounding the first evaluation region, calculating a brightness representative value and a saturation representative value in each of the first evaluation region and the reference region based on brightness values of the captured image and saturation values of color components thereof, and calculating the amount of generated gloss due to making up by calculating at least one of a difference between the brightness representative values and a difference between the saturation representative values in the first evaluation region and the reference region; a step of calculating the amount of stain portions by detecting the stain portions based on the brightness values or the color component values of the captured image; a step of calculating the amount of color irregularity portions by detecting the color irregularity portions based on the brightness values or the color component values of the captured image; a step of calculating the amount of wrinkle portions by detecting the wrinkle portions based on the brightness values of the captured image; a step of calculating the amount of pore portions by detecting the pore portions based on the brightness values or the color component values of the captured image; and a step of evaluating a feeling of visible moisture of the face of the subject for which making up is performed, using all of the amount of the generated gloss, the amount of stain portions, and the amount of color irregularity portions as the brightness-color indexes and using all of the amount of wrinkle portions and the amount of pore portions as the shape indexes, based on the brightness-color indexes and the shape indexes.

According to the invention, by calculating the amount of generated gloss, the amount of stain portions, and the amount of color irregularity portions as brightness-color indexes, calculating the amount of wrinkle portions and the amount of pore portions as shape indexes, and evaluating a feeling of visible moisture based on the brightness-color indexes and the shape indexes, it is possible to evaluate a feeling of visible moisture of a face of a subject for which making up is performed with high accuracy.

### BRIEF DESCRIPTION OF THE DRAWINGS

FIG. 1 is a block diagram illustrating a configuration of a moisture feeling evaluation device according to the invention.

FIG. 2 is a block diagram illustrating a configuration of a brightness-color index calculation unit.

FIG. 3 is a block diagram illustrating a configuration of a shape index calculation unit.

FIG. 4 is a diagram illustrating an evaluation region set in a face of a subject in a gloss calculation unit.

FIG. 5 is a diagram illustrating an evaluation region set in a face of a subject in a wrinkle calculation unit.

FIG. 6 is a graph illustrating a correlation between a total index value calculated using the moisture feeling evaluation device according to the invention and a visual evaluation value.

FIG. 7 is a graph illustrating a correlation between a total index value calculated using a moisture feeling evaluation device specific to the bare skin and a visual evaluation value.

### DESCRIPTION OF THE PREFERRED EMBODIMENTS

Hereinafter, embodiments of the invention will be described with reference to the accompanying drawings.

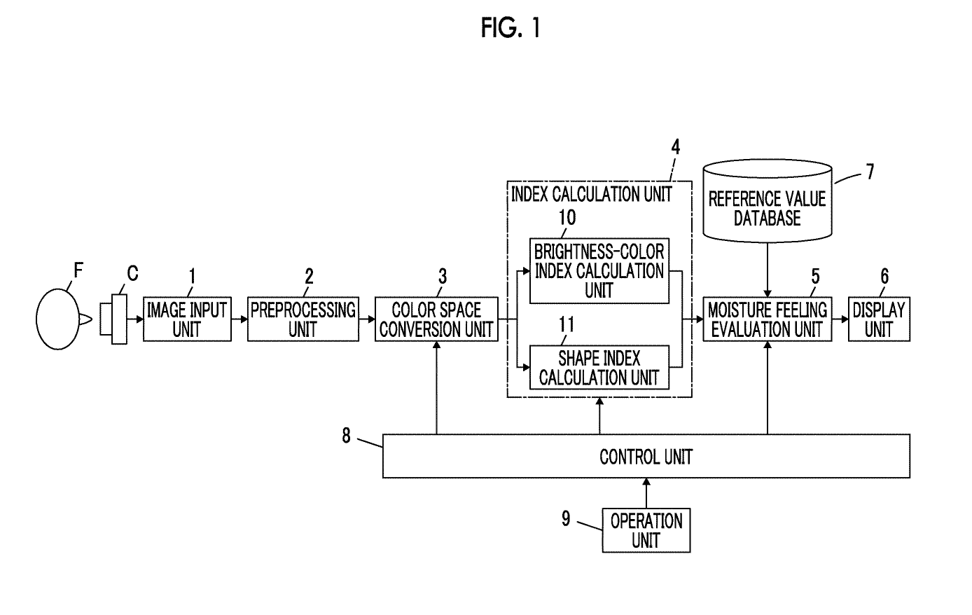

FIG. 1 shows a configuration of a moisture feeling evaluation device according to an embodiment of the invention. The moisture feeling evaluation device evaluates a feeling of visible moisture of a face F of a subject using a captured image obtained by imaging the face F of the subject from the front thereof using a camera C, and includes an image input unit 1 connected to the camera C. A preprocessing unit 2, a color space conversion unit 3, an index calculation unit 4, a moisture feeling evaluation unit 5 and a display unit 6 are sequentially connected to the image input unit 1. Further, a reference value database 7 is connected to the moisture feeling evaluation unit 5. In addition, a control unit 8 is connected to the color space conversion unit 3, the index calculation unit 4, and the moisture feeling evaluation unit 5, and an operation unit 9 is connected to the control unit 8.

The image input unit 1 receives an input of a captured image from the camera C that images the face F of the subject for which making up is performed (made-up skin).

Here, the making up performed for the face of the subject is to enhance gloss in cheek portions with respect to bare skin, and specifically, skin care products such as lotion, milk lotion, cream, oil, or essence may be used therefor. Further, the gloss refers to polish that evenly spreads over the entirety of the cheek portions. For example, a difference ΔC* between saturation average values of the cheek portions and regions around the cheek portions may be calculated, and when the difference ΔC* between saturation average values is equal to or higher than −2.5 and equal to or lower than −0.5, this may be defined as the gloss. The cheek portions refer to portions where the gloss is greatly enhanced when performing making up for the face of the subject. Further, the regions around the cheek portions refer to portions where the gloss is not nearly generated even though making up is performed for the face of the subject. Similar to a gloss calculation unit to be described later, each cheek portion may be set to a square region having side lengths of 3 cm, in which the center of an upper side thereof is disposed under about 1 cm from an outer corner of each eye (ear side end portion among both end portions of each eye), and the region around each cheek portion may be set to a square region having side lengths of 2 cm, in which a nose side end portion of an upper side thereof is disposed under about 1 cm from an inner corner of each eye (nose side end portion among both end portions of each eye).

Further, it is preferable that the captured image is an image obtained by imaging the face F of the subject from the front thereof so that both ears of the face F of the subject are included. Further, it is assumed that the captured image input from the camera C has an RGB color space. As the camera C, any camera capable of imaging the face F of the subject may be used, and for example, a digital camera, a CCD camera, or the like may be used. Further, a captured image obtained by imaging using a mobile phone such as a smart phone may be used.

The preprocessing unit 2 performs preprocessing such as light intensity correction and noise removal with respect to a captured image input through the image input unit 1.

The color space conversion unit 3 converts a color space of a captured image input from the preprocessing unit 2 to generate a color space converted image. As the color space converted image, an image of which the color space is converted into an L*a*b* color space, an LCH color space, an YCC color space, or the like may be used, for example. In a case where the color space is converted into the L*a*b* color space, a D65 light source may be used as a calculation light source. Further, the color space conversion unit 3 generates a brightness component image and a color component image by dividing the generated color space converted image into a brightness component (luminance component) and a color component, respectively. Specifically, in the case of a color space converted image having the L*a*b* color space, the brightness component corresponds to an L* component, the color component corresponds to an a* component (complementary component corresponding to red and green), a b* component (complementary component corresponding to yellow and blue), a C* component (chroma component), a Hue component (hue component), and the like.

The index calculation unit 4 includes a brightness-color index calculation unit 10 and a shape index calculation unit 11 which are respectively connected to the color space conversion unit 3.

The brightness-color index calculation unit 10 receives inputs of the brightness component image and the color component image from the color space conversion unit 3, respectively, and calculates a brightness-color index for evaluation of a feeling of visible moisture of the made-up skin based on brightness values of the brightness component image and color component values of the color component image.

The shape index calculation unit 11 receives inputs of the brightness component image and the color component image from the color space conversion unit 3, respectively, detects uneven portions of the made-up skin based on the brightness values of the brightness component image and the color component values of the color component image, and calculates a shape index for evaluation of the feeling of visible moisture of the made-up skin based on the amount of uneven portions.

The brightness-color index calculation unit 10 and the shape index calculation unit 11 output the respectively calculated brightness-color index and shape index to the moisture feeling evaluation unit 5.

The reference value database 7 stores a reference value of the feeling of visible moisture which is obtained in advance by visually evaluating faces of plural subjects for which making up is performed in association with the brightness-color index value and the shape index value. For example, with respect to the plural subjects for which making up is performed, the reference value of the feeling of visible moisture obtained thorough visual evaluation, the bright-color index value and the shape index value are calculated from the captured image, and a function indicating a reference value of the feeling of visible moisture with respect to a linear sum of the brightness-color index value and the shape index value may be calculated and stored.

The moisture feeling evaluation unit 5 evaluates a feeling of visible moisture of a face of a subject for which making up is performed based on the brightness-color index and the shape index.

Specifically, on the basis of the brightness-color index value calculated in the brightness-color index calculation unit 10 and the shape index value calculated in the shape index calculation unit 11, an evaluation value of the feeling of visible moisture is calculated with reference to the reference value database 7. For example, the moisture feeling evaluation unit 5 may evaluate the feeling of visible moisture on the basis of the function indicating the reference value of the feeling of visible moisture with respect to the linear sum of the brightness-color index value and the shape index value stored in the reference value database 7.

Here, the feeling of moisture shows a fresh and youthful skin which is tightened and transparent and contains moisture, and the moisture feeling evaluation unit 5 evaluates the feeling of visible moisture indicating a feeling of moisture with respect to the entire face F of the subject for which making up is performed.

The display unit 6 includes a display device such as an LCD, and displays an evaluation result of the feeling of visible moisture evaluated by the moisture feeling evaluation unit 5.

The operation unit 9 is a unit through which an operator performs an information input operation, and may be formed by a keyboard, a mouse, a track ball, a touch panel, or the like.

The control unit 8 controls the respective units in the moisture feeling evaluation device based on various command signals or the like input through the operation unit 9 by the operator.

The color space conversion unit 3, the index calculation unit 4, the moisture feeling evaluation unit 5, and the control unit 8 are configured by a CPU and an operation program that causes the CPU to perform various processes, but may be configured by a digital circuit. Further, a memory may be connected to the CPU through a signal line such as a bus, and for example, the captured image input to the image input unit 1, the brightness component image and the color component image generated in the color space conversion unit 3, the image generated in the index calculation unit 4, the evaluation result of the feeling of visible moisture calculated in the moisture feeling evaluation unit 5, and the like may be respectively stored in the memory. Further, the images stored in the memory and the evaluation result of the feeling of visible moisture may be displayed on the display unit 6 under the control of the control unit 8.

Next, the brightness-color index calculation unit 10 of the index calculation unit 4 will be described in detail.

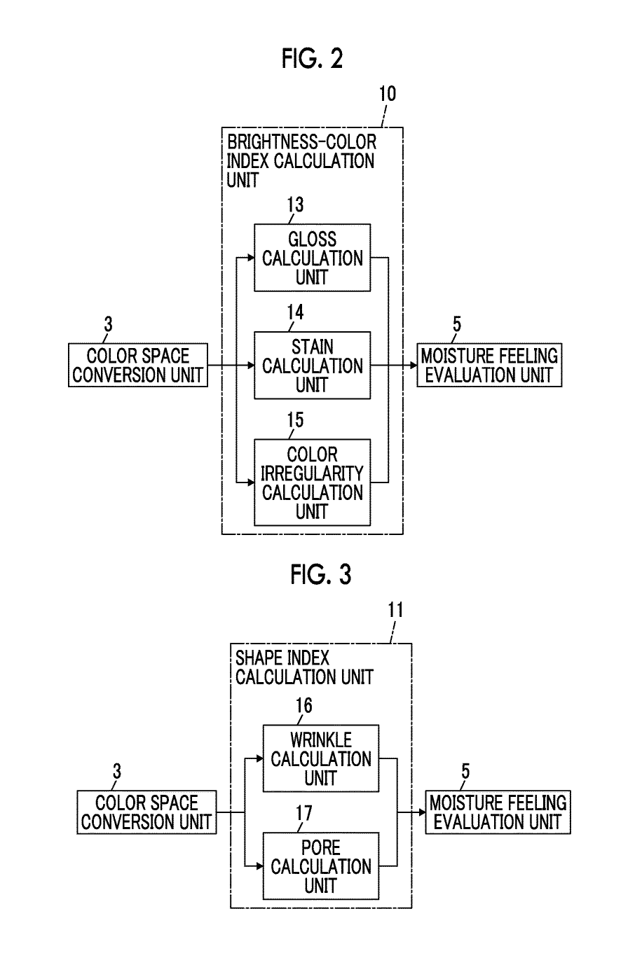

As shown in FIG. 2, the brightness-color index calculation unit 10 includes a gloss calculation unit 13, and a stain calculation unit 14, and a color irregularity calculation unit 15 which are respectively connected to the color space conversion unit 3 and the moisture feeling evaluation unit 5.

The gloss calculation unit 13 calculates the amount of generated gloss in the made-up skin as a brightness-color index. Specifically, the gloss calculation unit 13 sets evaluation regions R1 in cheek portions, and sets reference regions R1a in portions surrounding the evaluation regions R1, with respect to the brightness component image and the color component image generated in the color space conversion unit 3. Subsequently, the gloss calculation unit 13 calculates brightness representative values of the evaluation regions R1 and saturation representative values of the color components, and calculates a difference between the brightness representative values and a difference between the saturation representative values in the evaluation regions R1 and the reference regions R1a, to thereby make it possible to calculate the amount of gloss generated based on making up and to set the amount of generated gloss as the brightness-color index.

Here, a brightness average value, a brightness maximum value, and the like may be used as a brightness representative value, and a saturation average value, a saturation minimum value, and the like may be used as a saturation representative value. That is, the gloss calculation unit 13 may calculate a difference between brightness average values and a difference between saturation average values, a difference between brightness maximum values and a difference between saturation minimum values, or the like as the amount of generated gloss, for example, in the evaluation regions R1 and the reference regions R1a.

The stain calculation unit 14 calculates the amount of stain portions in the made-up skin as a brightness-color index. Here, the stain portion refers to a portion where the brightness value or the color component value is locally changed, having a predetermined size, for example, a size (a maximum width or a diameter of a circumscribed circle) which is larger than 2 mm and smaller than 50 mm.

Specifically, the stain calculation unit 14 sets an evaluation region R2 with respect to the brightness component image and the color component image generated in the color space conversion unit 3. The evaluation region R2 may be set with respect to the entire face F or a cheek portion of the subject, for example. Subsequently, the stain calculation unit 14 detects stain portions where the brightness value or the color component value is locally changed, having a predetermined size, from the evaluation region R2, and for example, calculates a total area of the stain portions in the evaluation region R2, an area ratio of the stain portions with respect to the evaluation region R2, shades of the stain portions based on the intensity of brightness, or the number of stain portions in the evaluation region R2, as the brightness-color index.

The color irregularity calculation unit 15 calculates the amount of color irregularity portions in the made-up skin as a brightness-color index. Here, the color irregularity portion refers to a portion where the brightness value or the color component value is locally changed, its size is larger than that of the stain portion, and its boundary is unclear.

Specifically, the color irregularity calculation unit 15 sets an evaluation region R3 with respect to the brightness component image and the color component image generated in the color space conversion unit 3. The evaluation region R3 may be set with respect to the entire face F or a cheek portion of the subject, for example. Subsequently, the color irregularity calculation unit 15 detects a color irregularity portion where the brightness value or the color component value is locally changed and its size is larger than that of the stain portion from the evaluation region R3, and for example, calculates a total area of the color irregularity portions in the evaluation region R3, an area ratio of the color irregularity portions with respect to the evaluation region R3, a shade of the color irregularity portion based on the intensity of brightness, or the number of color irregularity portions in the evaluation region R3, as the brightness-color index.

Next, the shape index calculation unit 11 of the index calculation unit 4 will be described in detail.

As shown in FIG. 3, the shape index calculation unit 11 includes a wrinkle calculation unit 16 and a pore calculation unit 17 which are respectively connected to the color space conversion unit 3 and the moisture feeling evaluation unit 5.

The wrinkle calculation unit 16 calculates the amount of wrinkle portions in the made-up skin as a shape index. Here, the wrinkle portion refers to a portion where the brightness value or the color component value is locally changed and a shape which elongatedly extends in a predetermined direction is formed.

Specifically, the wrinkle calculation unit 16 sets an evaluation region R4 that extends from the nostrils to the corners of the mouth with respect to the brightness component image generated in the color space conversion unit 3. Subsequently, the wrinkle calculation unit 16 detects wrinkle portions where the brightness decreases in the evaluation region R4, and for example, calculates a total area of the wrinkle portions in the evaluation region R4, an area ratio of the wrinkle portions with respect to the evaluation region R4, shades of the wrinkle portions based on the intensity of brightness, or the lengths of the wrinkle portions in the evaluation region R4, as the shape index.

The pore calculation unit 17 calculates the amount of pore portions in the made-up skin as a shape index. Here, the pore portion refers to a portion where the brightness value or the color component value is locally changed and its size is smaller than that of the stain portion.

Specifically, the pore calculation unit 17 sets an evaluation region R5 with respect to the brightness component image and the color component image generated in the color space conversion unit 3. The evaluation region R5 may be set with respect to the entire face F or a cheek portion of the subject, for example. Subsequently, the pore calculation unit 17 detects pore portions where the brightness component value or the color component value is locally changed and its size is smaller than that of the stain portion from the evaluation region R5, and for example, calculates a total area of the pore portions in the evaluation region R5, an area ratio of the pore portions with respect to the evaluation region R5, a shade of the pore portion based on the intensity of brightness, or the number of pore portions in the evaluation region R5, as the shape index.

Next, an operation of this embodiment will be described.

First, a captured image obtained by imaging the face F of a subject for which making up is performed using the camera C is input to the preprocessing unit 2 through the image input unit 1 of the moisture feeling evaluation device from the camera C, as shown in FIG. 1. The captured image is subject to preprocessing such as light source correction or noise removal, and is output to the color space conversion unit 3 from the preprocessing unit 2. Then, a color space of the captured image is converted into the L*a*b* color space by the color space conversion unit 3, for example, so that a color space converted image is generated. Further, the color space conversion unit 3 extracts a brightness component and a color component from the color space converted image, and generates a brightness component image and a color component image, respectively. For example, the color space conversion unit 3 may generate an L* component image as the brightness component image, and a C* component image, an a* component image, and a b* component image as the color component image.

The color space conversion unit 3 outputs the generated brightness component image and color component image to the gloss calculation unit 13, the stain calculation unit 14, and the color irregularity calculation unit 15 of the brightness-color index calculation unit 10, respectively.

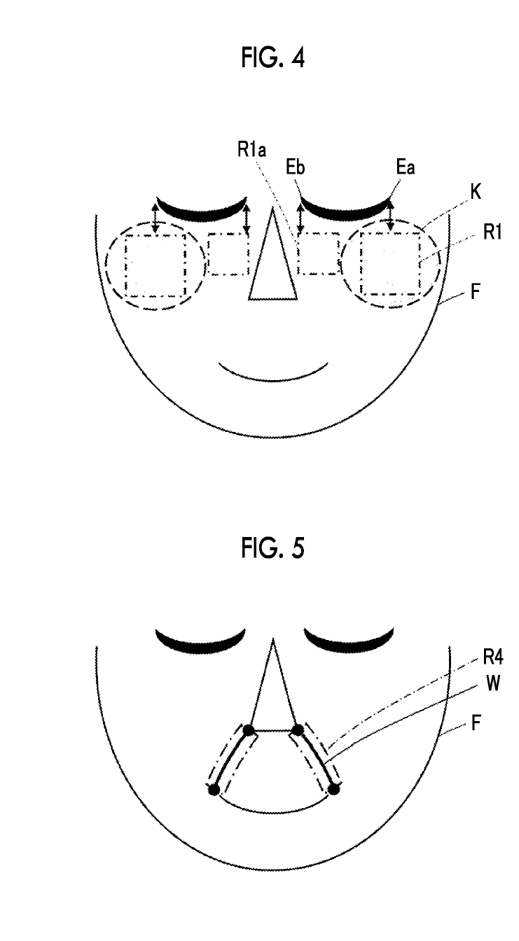

The gloss calculation unit 13 sets the evaluation regions R1 in the cheek portions of the face F of the subject with respect to the brightness component image and the color component image generated in the color space conversion unit 3, and sets the evaluation regions R1 in portions surrounding the evaluation regions. For example, the gloss calculation unit 13 may set the evaluation regions R1 and the reference regions R1a, respectively, with respect to the L* component image and the C* component image, as shown in FIG. 4.

Each evaluation region R1 may be set as a square region having side lengths of 3 cm, in which the center of an upper side thereof is disposed under about 1 cm from an outer corner of each eye Ea (ear side end portion among both end portions of each eye), for example. Further, each reference region R1a may be set as a square region having side lengths of 2 cm, in which a nose side end portion of an upper side thereof is disposed under about 1 cm from an inner corner of each eye Eb (nose side end portion among both end portions of each eye).

Generally, if making up is performed with respect to the face of a subject using a skin care product or the like, gloss of a cheek portion K is greatly enhanced compared with the bare skin before making up. That is, gloss generated in the cheek portion K greatly change between the bare skin and the made-up skin. Thus, by comparing the evaluation region R1 where gloss is greatly enhanced due to makeup with the reference region R1a where a gloss change due to making up is relatively small, it is possible to calculate the amount of generated gloss due to making up.

Specifically, the gloss calculation unit 13 calculates an average value of L* values and an average value of C* values in the evaluation region R1, and an average value of L* values and an average value of C* values in the reference region R1a, based on L* values of the brightness component image and C* values of the color component image. Subsequently, the gloss calculation unit 13 calculates a difference ΔL* between the average value of the L* values in the evaluation region R1 and the average value of the L* values in the reference region R1a as the amount of generated gloss due to making up. Similarly, the gloss calculation unit 13 calculates a difference ΔC* between the average value of the C* values in the evaluation region R1 and the average value of the C* values in the reference region R1a as the amount of generated gloss due to making up.

The amount of generated gloss due to making up greatly affects a feeling of moisture (feeling of visual moisture) when generally viewing the face F of the subject. However, since the amount of gloss generated in the cheek portion K is small in the bare skin, in a moisture feeling evaluation method specific to the bare skin, the amount of gloss does not greatly affect an evaluation result, and thus, it is not possible to evaluate the feeling of visual moisture of the made-up skin with high accuracy. Thus, by adding the amount of generated gloss calculated as described above to an evaluation index of the feeling of visual moisture as a brightness-color index, it is possible to evaluate the feeling of visual moisture of the made-up skin with high accuracy.

Here, with respect to the amount of generated gloss, the feeling of visual moisture of the made-up skin is more strongly felt as the ΔL* value becomes higher and the ΔC* value becomes lower. Thus, the gloss calculation unit 13 outputs the ΔL* value and the ΔC* value to the moisture feeling evaluation unit 5 as a brightness-color index.

Here, in a case where the ΔL* value is excessively high and the ΔC* value is excessively low, unnaturally strong gloss, namely, shine, may cause deterioration in the feeling of visual moisture. Thus, it is preferable that the ΔL* value is equal to or smaller than 5 and the ΔC* value is equal to or greater than −2.5.

The stain calculation unit 14 sets the evaluation region R2 with respect to the brightness component image or the color component image generated in the color space conversion unit 3, and detects stain portions from the evaluation region R2. For example, the stain calculation unit 14 may set the evaluation region R2 in a cheek portion of the face F of the subject with respect to the L* component image.

Here, the stain portions may be detected by generating a difference-of-Gaussian (Dog) image, for example. Specifically, Dog images having different Gaussian sizes are generated from an L* component image. Generally, a stain has a diameter of a circumscribed circle of 2 mm or longer and shorter than 10 mm, and a frequency of 0.05 cycles/mm to 0.25 cycles/mm. The stain calculation unit 14 performs Dog image processing so that a component having a stain frequency band is extracted. Further, when performing the Dog image processing, the stain calculation unit 14 may calculate a shape of each component from a binary image which is subject to threshold value processing, and may detect a component having a round shape and a circularity (4π×area)/circumference² of 0.4 to 1.0, preferably 0.6 to 1.0, and having a circumferential length of 2 mm or longer and shorter than 10 mm as the stain portions.

Further, the stain portions may be detected by extracting a component of which a redness value and a yellowness value are smaller than a predetermined threshold value after performing the above-mentioned Dog image processing. For example, the stain calculation unit 14 may set the evaluation region R2 in a region where stains are easily generated, may set a reference region R2a in a region where stains are not easily generated, may calculate a difference Δa* between a* component values or a difference Δb* between b* component values in the evaluation region R2 and the reference region R2a, and may extract a portion where the Δa* value is smaller than 2.5 or a portion where Δb* value is smaller than 2.5 as a stain portion.

The stain calculation unit 14 may generate a Dog image using color component mages such as an a* component image and a b* component image, in addition to the L* component image, to thereby detect the stain portions in a similar way to the above-described method. Further, the stain calculation unit 14 may generate a Dog image using a B channel in the RGB color space to detect the stain portions.

In addition, the stain calculation unit 14 may not generate a Dog image, and for example, may extract a component having a strength which is equal to or smaller than a predetermined threshold value from the L* component image, and may perform main component analysis and independent component analysis with respect to the extracted component, for example, to detect the stain portions.

Further, the stain calculation unit 14 calculates a total area of stain portions detected in the evaluation region R2 as the brightness-color index, for example. Here, the stain portion refers to a portion where the face F of the subject for which making up is performed generally shows a dark impression and the feeling of visible moisture of the face F of the subject for which making up is performed becomes higher as the total area becomes smaller.

The stain calculation unit 14 outputs the calculated brightness-color index to the moisture feeling evaluation unit 5.

The color irregularity calculation unit 15 sets the evaluation region R3 with respect to the brightness component image or the color component image generated in the color space conversion unit 3, and detects color irregularity portions from the evaluation region R3. For example, the color irregularity calculation unit 15 may set the evaluation region R3 in a cheek portion of the face F of the subject with respect to the L* component image.

Here, the color irregularity portion may be detected by generating a Dog image, in a similar way to the detection of the stain portion. That is, Dog images having different Gaussian sizes are generated from an L* component image. Generally, a color irregularity has a size of which a diameter of a circumscribed circle is about 10 mm or greater, and has a frequency of 0.05 cycles/mm or greater. The color irregularity calculation unit 15 performs Dog image processing so that a component having a color irregularity frequency band is extracted. Further, when performing the Dog image processing, the color irregularity calculation unit 15 may calculate a shape of each component from a binary image which is subject to threshold value processing, and may detect a component having a round shape and a circularity (4π×area)/circumference² of 0.4 to 1.0, preferably, 0.6 to 1.0, and having a circumferential length of about 10 mm or longer.

Further, the color irregularity portion may be detected by extracting a component of which a redness value and a yellowness value are smaller than a predetermined threshold value after performing the above-mentioned Dog image processing. For example, the color irregularity calculation unit 15 may set the evaluation region R3 in a region where color irregularities are easily generated, may set a reference region R3a in a region where color irregularities are not easily generated, may calculate a difference Δa* between a* component values or a difference Δb* between b* component values in the evaluation region R3 and the reference region R3a, and may extract a portion where the Δa* value is smaller than 2.5 or a portion where Δb* value is smaller than 2.5 as a stain portion.

The color irregularity calculation unit 15 may generate a Dog image using color component images such as an a* component image or a b* component image, in addition to the L* component image, to thereby detect color irregularity portions in a similar way to the above-described method. Further, the color irregularity calculation unit 15 may generate a Dog image using a B channel in the RGB color space to detect the color irregularity portions.

In addition, the color irregularity calculation unit 15 calculates a total area of color irregularity portions detected in the evaluation region R3 as the brightness-color index, for example. Here, the color irregularity portion refers to a portion where the face F of the subject for which making up is performed generally shows a dark impression, in which the feeling of visible moisture of the face F of the subject for which making up is performed becomes higher as the total area becomes smaller.

The color irregularity calculation unit 15 outputs the calculated brightness-color index to the moisture feeling evaluation unit 5.

Further, the color space conversion unit 3 outputs the calculated brightness value and color component value to the wrinkle calculation unit 16 and the pore calculation unit 17 of the shape index calculation unit 11, respectively.

The wrinkle calculation unit 16 sets the evaluation region R4 that extends from the nostrils to the corners of the mouth with respect to the brightness component image or the color component image generated in the color space conversion unit 3, as shown in FIG. 5, and detects a wrinkle portion W from the evaluation region R4. Further, the wrinkle calculation unit 16 sets a reference region R4a around the evaluation region R4, and calculates an average value of L* values in the reference region R4a.

Subsequently, the wrinkle calculation unit 16 creates an ΔL* component image obtained by subtracting the average value of the L* values in the reference region R4a from the L* values in the evaluation region R4, and detects a wrinkle portion from the evaluation region R4 of the ΔL* component image based on a predetermined threshold value which is set in advance. For example, a portion where the ΔL* value is smaller than 10 in the evaluation region R4 may be detected as a wrinkle portion.

Further, the wrinkle calculation unit 16 calculates a total area of wrinkle portions detected in the evaluation region R4 as the shape index, for example. Here, the wrinkle portion detected in the evaluation region R4 refers to a portion which is a so-called nasolabial fold, in which the face F of the subject for which making up is performed generally shows an impression as an uneven portion where shadow is generated and the feeling of visible moisture of the face F of the subject for which making up is performed becomes higher as the total area becomes smaller.

The wrinkle calculation unit 16 outputs the calculated shape index to the moisture feeling evaluation unit 5.

The pore calculation unit 17 sets the evaluation region R5 with respect to the brightness component image or the color component image generated in the color space conversion unit 3, and detects pore portions from the evaluation region R5. For example, the pore calculation unit 17 may set the evaluation region R5 in a cheek portion of the face F of the subject with respect to the L* component image.

Here, the pore portions may be detected by generating a Dog image, in a similar way to the case where the stain portions are detected. That is, Dog images having different Gaussian sizes are generated from an L* component image. Generally, a pore has a diameter of a circumscribed circle of 0.5 mm or longer and shorter than 2 mm, and a frequency of 0.25 cycles/mm to 1.0 cycles/mm. The pore calculation unit 17 performs Dog image processing so that a component having a pore frequency band is extracted. Further, when performing the Dog image processing, the pore calculation unit 17 may calculate a shape of each component from a binary image which is subject to threshold value processing, and may detect a component having a round shape and a circularity (4π×area)/circumference² of 0.4 to 1.0, preferably, 0.6 to 1.0, and having a circumferential length of 0.5 mm or longer and shorter than 2 mm as the pore portions.

Further, the pore portions may be detected by extracting a component of which a redness value and a yellowness value are smaller than a predetermined threshold value after performing the above-mentioned Dog image processing. For example, the pore calculation unit 17 may set the evaluation region R5 in a region where a large amount of pore portions are present, may set a reference region R5a in a region where a small amount of pore portions are present, may calculate a difference Δa* between a* component values or a difference Δb* between b* component values in the evaluation region R5 and the reference region R5a, and may extract a portion where the Δa* value is smaller than 2.5 or a portion where the Δb* value is smaller than 2.5 as a stain portion.

The pore calculation unit 17 may generate a Dog image using color component images such as an a* component image and a b* component image, in addition to the L* component image, to thereby detect the pore portions in a similar way to the above-described method. Further, the pore calculation unit 17 may generate a Dog image using a B channel in the RGB color space to detect the pore portions.

In addition, the pore calculation unit 17 calculates a total area of pore portions detected in the evaluation region R5 as the shape index, for example. Here, the pore portion refers to a portion where the face F of the subject for which making up is performed generally shows an impression which is an uneven portion which generates a shadow, in which the feeling of visible moisture of the face F of the subject for which making up is performed becomes higher as the total area becomes smaller.

The pore calculation unit 17 outputs the calculated shape index to the moisture feeling evaluation unit 5.

In this way, all the brightness-color indexes which are calculated in the gloss calculation unit 13, the stain calculation unit 14, and the color irregularity calculation unit 15 of the brightness-color index calculation unit 10, and all the shape indexes which are calculated in the wrinkle calculation unit 16 and the pore calculation unit 17 of the shape index calculation unit 11 are input to the moisture feeling evaluation unit 5.

The moisture feeling evaluation unit 5 makes reference to the reference value database 7 based on the input brightness-color index values and shape index values. In the reference value database 7, a reference value of a feeling of visible moisture obtained by performing visual evaluation in advance is stored with respect to a total index obtained by linearly summing the brightness-color index values and the shape index values using a multiple regression equation or the like. Thus, the moisture feeling evaluation unit 5 calculates the reference value of the feeling of visible moisture depending on the brightness-color index values and the shape index values input from the index calculation unit 4, with reference to the reference value database 7, and evaluates the feeling of visible moisture of the face F of the subject for which making up is performed based on the reference value.

The evaluation result of the feeling of visible moisture calculated by the moisture feeling evaluation unit 5 is output to the display unit 6 and is displayed thereon.

According to this embodiment, since respective physical characteristics of the feeling of visible moisture over the entire face F of the subject for which making up is performed is evaluated in a complex manner, it is possible to perform evaluation close to a feeling when generally viewing the face F of the subject for which making up is performed, and to evaluate the feeling of visible moisture with high accuracy. Further, the amount of generated gloss calculated in the gloss calculation unit 13 is an evaluation index specific to the made-up skin indicating a change of the feeling of visible moisture due to making up, and it is possible to evaluate the feeling of visible moisture of the made-up skin with high accuracy using the evaluation index.

The above-described evaluation of the feeling of visible moisture may be executed by operating a computer configured by input means, a CPU, a memory, and the like by a moisture feeling evaluation program. That is, by operating the computer by the moisture feeling evaluation program, the image input unit 1 acquires a captured image obtained by imaging a face of a subject, and the CPU executes the preprocessing unit 2, the color space conversion unit 3, the index calculation unit 4, and the moisture feeling evaluation unit 5, to thereby perform evaluation of the feeling of visible moisture with respect to the face of the subject.

Further, in the above-described embodiment, a configuration in which an image captured from the camera C connected to the image input unit 1 is input, but the invention is not limited thereto, and any configuration capable of inputting a captured image may be used.

For example, a captured image may be input to the image input unit 1 through a network from a computer which retains the captured image. The moisture feeling evaluation device evaluates a feeling of visible moisture based on the captured image input from the computer, and stores the evaluation result in a server or the like. Thus, a user is able to browse the evaluation result of the feeling of visible moisture by accessing the server, or to acquire the evaluation result of the feeling of visible moisture through the network from the server.

In addition, in the above-described embodiment, the gloss calculation unit 13 calculates a difference between brightness representative values and a difference between saturation representative values in the first evaluation region R1 and the reference region R1a to calculate the amount of generated gloss, but may more accurately show the amount of generated gloss by adding new evaluation relating to gloss.

For example, the gloss calculation unit 13 may newly add uniformity of brightness values and uniformity of saturation values in the first evaluation region R1 to the evaluation of the amount of generated gloss. Specifically, by respectively performing fast Fourier transform (FFT) with respect to a brightness component image and a color component image, the gloss calculation unit 13 may calculate a brightness frequency characteristic and a saturation frequency characteristic in the first evaluation region R1, and may calculate the uniformity of the brightness values and the uniformity of the saturation values based on the frequency characteristics. By newly adding the uniformity of the brightness values and the uniformity of the saturation values, it is possible to more accurately show the amount of generated gloss.

Further, the gloss calculation unit 13 may newly add spread of brightness and spread of saturation in the first evaluation region R1 to the evaluation of the amount of generated gloss. Specifically, the gloss calculation unit 13 may respectively profile a brightness component image and a color component image in one dimension, to thereby calculate a half-value width of brightness values as spread of brightness and a half-value width of saturation values as spread of saturation. By newly adding the half-value width of the brightness values and the half-value width of the saturation values, it is possible to more accurately show the amount of generated gloss.

Furthermore, in the above-described embodiment, the gloss calculation unit 13 calculates both of the difference between the brightness representative values and the difference between the saturation representative values in the first evaluation region R1 and the reference region R1a to calculate the amount of generated gloss, but the gloss calculation unit 13 may calculate only one of the difference between the brightness representative values and the difference between the saturation representative values in the first evaluation region R1 and the reference region R1a to thereby calculate the amount of generated gloss.

For example, the gloss calculation unit 13 may calculate a saturation average value and a saturation minimum value with respect to each of the first evaluation region R1 and the reference region R1a, may calculate a difference between the saturation average values or a difference between the saturation minimum average values in the first evaluation region R1 and the reference region R1a as the difference between the saturation representative values, and may set only the difference between the saturation representative values as the amount of generated gloss. Further, the gloss calculation unit 13 may calculate a brightness average value and a brightness maximum value with respect to each of the first evaluation region R1 and the reference region R1a, may calculate a difference between the brightness average values or a difference between the brightness maximum average values in the first evaluation region R1 and the reference region R1a as the difference between the brightness representative values, and may set only the difference between the brightness representative values as the amount of generated gloss.

In a case where one of the difference between the brightness representative values and the difference between the saturation representative values in the first evaluation region R1 and the reference region R1a is calculated as the amount of generated gloss, it is preferable that the difference between the saturation representative values is calculated as the amount of generated gloss.

In reality, an example in which a feeling of visible moisture of a face of a subject for which making up is performed is evaluated using the moisture feeling evaluation device will be described.

In this example, a feeling of visible moisture was evaluated using the moisture feeling evaluation device with respect to subjects in their twenties to fifties, and visual evaluation of the feeling of visible moisture when plural observers generally view the face F of the subject was performed.

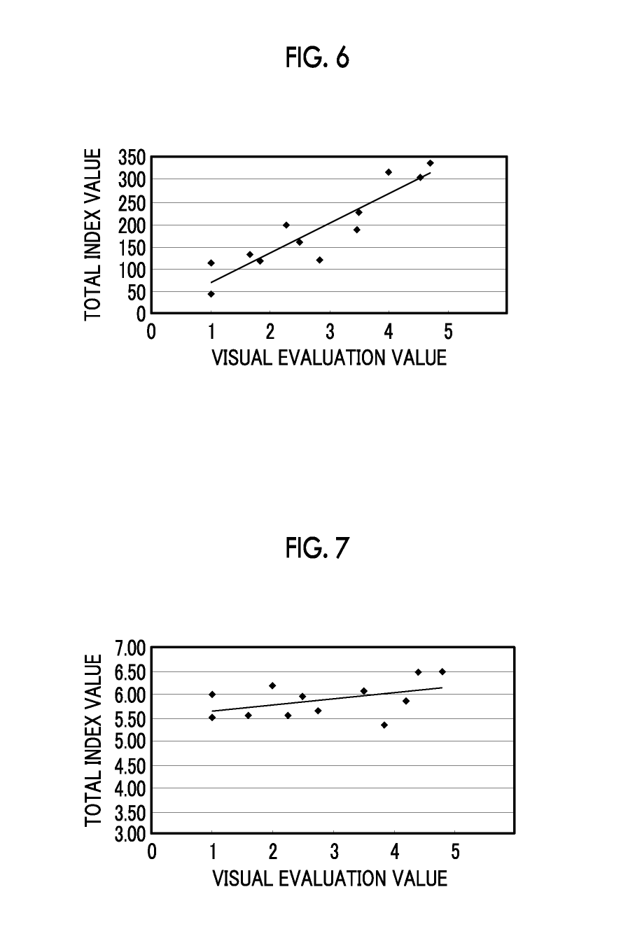

FIG. 6 is a graph obtained by plotting a total index value calculated by linearly summing brightness-color indexes and shape indexes obtained using the moisture feeling evaluation device with respect to a visual evaluation value. Here, the visual evaluation value is an average value obtained by evaluating the feeling of visible moisture over five stages by visual evaluation of plural observers, in which it is evaluated that the feeling of visible moisture becomes higher as the value becomes closer to 5.

As a result of calculating a correlation between the total index value and the visual evaluation value with respect to FIG. 6, a correlation coefficient R² was 0.84.

In this way, by evaluating respective physical characteristics of the feeling of visible moisture over the entire face F of the subject for which making up is performed in a complex manner, it can be understood that a correlation with visual evaluation when generally observing the face F of the subject is considerably high, and thus, it is possible to evaluate the feeling of visible moisture with high accuracy.

Further, FIG. 7 shows a comparative example in which a feeling of visual moisture of the face of a subject for which making up is performed is evaluated using a moisture feeling evaluation device specific to the bare skin. The moisture feeling evaluation device is an evaluation device disclosed in JP2014-031460. Specifically, the moisture feeling evaluation device calculates the brightness of the skin, the amount of dullness portions, the amount of stain portions, the amount of color irregularity portions, the amount of wrinkle portions, the amount of pore portions, and the amount of cavities generated in a cheek outline shape. FIG. 7 is a graph formed by plotting a total index value obtained by measuring the made-up skin using the evaluation device with respect to a visual evaluation value.

In FIG. 7, as a result obtained by calculating a correlation between a total index value and a visual evaluation value, a correlation coefficient R² was 0.20.

From the comparison example, it can be understood that it is not possible to obtain a high correlation with visual evaluation even if the made-up skin is evaluated using the same evaluation index as in the bare skin, and thus, by newly adding the amount of generated gloss specific to the made-up skin as an evaluation index, it is possible to more accurately evaluate a feeling of visual moisture of the made-up skin.

### EXPLANATION OF REFERENCES

1: image input unit

2: preprocessing unit

3: color space conversion unit

4: index calculation unit

5: moisture feeling evaluation unit

6: display unit

7: reference value database

8: control unit

9: operation unit

10: brightness-color index calculation unit

11: shape index calculation unit

13: gloss calculation unit

14: stain calculation unit

15: color irregularity calculation unit

16: wrinkle calculation unit

17: pore calculation unit

R1, R4: evaluation region

R1a: reference region

F: face

C: camera

K: cheek portion

Ea: outer corner of eye

Eb: inner corner of eye

W: wrinkle portion

What is claimed is:

1. A moisture feeling evaluation device comprising:
an image input unit that receives an input of a captured image obtained by imaging a face of a subject for which making up is performed;
a brightness-color index calculation unit that calculates a brightness-color index of the face of the subject for which making up is performed based on brightness values and color component values of the captured image input to the image input unit;
a shape index calculation unit that detects uneven portions of the face of the subject for which making up is performed based on the brightness values and the color component values of the captured image input to the image input unit and calculates a shape index based on the amount of uneven portions; and
a moisture feeling evaluation unit that evaluates a feeling of visible moisture of the face of the subject for which making up is performed based on the brightness-color index and the shape index,
wherein the brightness-color index calculation unit includes a gloss calculation unit that calculates the amount of generated gloss due to making up by setting a first evaluation region in a cheek portion of the face of the subject and setting a reference region in a portion surrounding the first evaluation region and calculating at least one of a difference between brightness representative values and a difference between saturation representative values of the color components in the first evaluation region and the reference region, a stain calculation unit that calculates the amount of stain portions by detecting the stain portions based on the brightness values or the color component values, and a color irregularity calculation unit that calculates the amount of color irregularity portions by detecting the color irregularity portions based on the brightness values or the color component values, and calculates all of the amount of the generated gloss, the amount of stain portions, and the amount of color irregularity portions as the brightness-color indexes, and
wherein the shape index calculation unit includes a wrinkle calculation unit that calculates the amount of wrinkle portions by detecting the wrinkle portions based on the brightness values, a pore calculation unit that calculates the amount of pore portions by detecting the pore portions based on the brightness values or the color component values, and calculates all of the amount of wrinkle portions and the amount of pore portions as the shape indexes.

2. The moisture feeling evaluation device according to claim 1,
wherein the gloss calculation unit calculates a difference between saturation average values or a difference between saturation minimum values in the first evaluation region and the reference region as the difference between the saturation representative values by calculating a saturation average value or a saturation minimum value with respect to each of the first evaluation region and the reference region.

3. The moisture feeling evaluation device according to claim 1,
wherein the gloss calculation unit calculates a difference between brightness average values or a difference between brightness maximum values in the first evaluation region and the reference region as the difference between the brightness representative values by calculating a brightness average value or a brightness maximum value with respect to each of the first evaluation region and the reference region.

4. The moisture feeling evaluation device according to claim 2,
wherein the stain calculation unit sets a second evaluation region in the face of the subject, detects the stain portions where the brightness value or the color component value is locally changed, having a predetermined size, from the second evaluation region, and calculates at least one of a total area of the stain portions, an area ratio of the stain portions with respect to the second evaluation region, shades of the stain portions, or the number of stain portions in the second evaluation region, as the amount of stain portions, and
wherein the color irregularity calculation unit sets a third evaluation region in the face of the subject, detects the color irregularity portions where the brightness value or the color component value is locally changed, having a size larger than that of each stain portion, from the third evaluation region, and calculates at least one of a total area of the color irregularity portions, an area ratio of the color irregularity portions with respect to the third evaluation region, shades of the color irregularity portions, or the number of color irregularity portions in the third evaluation region, as the amount of color irregularity portions.

5. The moisture feeling evaluation device according to claim 4,
wherein the wrinkle calculation unit sets a fourth evaluation region that extends from the nostrils to the corners of the mouth in the face of the subject, detects the wrinkle portions where the brightness value decreases in the fourth evaluation region, and calculates at least one of a total area of the wrinkle portions, an area ratio of the wrinkle portions with respect to the fourth evaluation region, shades of the wrinkle portions, or the lengths of the wrinkle portions, as the amount of wrinkle portions, and
wherein the pore calculation unit sets a fifth evaluation region in the face of the subject, detects the pore portions where the brightness value or the color component value is locally changed, having a size smaller than that of each stain portion, from the fifth evaluation region, and calculates at least one of a total area of the pore portions, an area ratio of the pore portions with respect to the fifth evaluation region, shades of the pore portions, or the number of pore portions in the fifth evaluation region, as the amount of pore portions.

6. The moisture feeling evaluation device according to claim 1,
wherein the moisture feeling evaluation unit calculates in advance a reference value of the feeling of visible moisture with respect to a linear sum of the brightness-color index and the shape index by visually evaluating faces of a plurality of subjects for which making up is performed, and evaluates the feeling of visible moisture based on the reference value.

7. The moisture feeling evaluation device according to claim 6, further comprising:
a database that stores the reference value with respect to the linear sum of the brightness-color index and the shape index,
wherein the moisture feeling evaluation unit calculates an evaluation value of the feeling of visible moisture with reference to the database based on the brightness-color index value calculated in the brightness-color index calculation unit and the shape index value calculated in the shape index calculation unit.

8. A moisture feeling evaluation method comprising:
receiving an input of a captured image obtained by imaging a face of a subject for which making up is performed;
setting a first evaluation region in a cheek portion of the face of the subject, setting a reference region in a portion surrounding the first evaluation region, calculating a brightness representative value and a saturation representative value in each of the first evaluation region and the reference region based on brightness values of the captured image and saturation values of color components thereof, and calculating the amount of generated gloss due to making up by calculating at least one of a difference between the brightness representative values and a difference between the saturation representative values in the first evaluation region and the reference region;
calculating the amount of stain portions by detecting the stain portions based on the brightness values or the color component values of the captured image;
calculating the amount of color irregularity portions by detecting the color irregularity portions based on the brightness values or the color component values of the captured image;
calculating the amount of wrinkle portions by detecting the wrinkle portions based on the brightness values of the captured image;
calculating the amount of pore portions by detecting the pore portions based on the brightness values or the color component values of the captured image; and
evaluating a feeling of visible moisture of the face of the subject for which making up is performed, using all of the amount of the generated gloss, the amount of stain portions, and the amount of color irregularity portions as the brightness-color indexes and using all of the amount of wrinkle portions and the amount of pore portions as the shape indexes, based on the brightness-color indexes and the shape indexes.

9. A non-transitory computer-readable recording medium that records a moisture feeling evaluation program that causes a computer to execute:
a step of receiving an input of a captured image obtained by imaging a face of a subject for which making up is performed;
a step of setting a first evaluation region in a cheek portion of the face of the subject, setting a reference region in a portion surrounding the first evaluation region, calculating a brightness representative value and a saturation representative value in each of the first evaluation region and the reference region based on brightness values of the captured image and saturation values of color components thereof, and calculating the amount of generated gloss due to making up by calculating at least one of a difference between the brightness representative values and a difference between the saturation representative values in the first evaluation region and the reference region;
a step of calculating the amount of stain portions by detecting the stain portions based on the brightness values or the color component values of the captured image;
a step of calculating the amount of color irregularity portions by detecting the color irregularity portions based on the brightness values or the color component values of the captured image;
a step of calculating the amount of wrinkle portions by detecting the wrinkle portions based on the brightness values of the captured image;
a step of calculating the amount of pore portions by detecting the pore portions based on the brightness values or the color component values of the captured image; and
a step of evaluating a feeling of visible moisture of the face of the subject for which making up is performed, using all of the amount of the generated gloss, the amount of stain portions, and the amount of color irregularity portions as the brightness-color indexes and using all of the amount of wrinkle portions and the amount of pore portions as the shape indexes, based on the brightness-color indexes and the shape indexes.
